(19) United States
(12) Patent Application Publication
(10) Pub. No.: US 2008/0194949 A1
(43) Pub. Date: Aug. 14, 2008

(54) Surgical Positioning Device

(76) Inventors: Been-Der Yang, Taipei (TW); Jaw-Lin Wang, Taipei (TW); Yao-Hung Wang, Taipei (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/019,778

(22) Filed: Jan. 25, 2008

(30) Foreign Application Priority Data
Feb. 9, 2007 (TW) 096104898

(51) Int. Cl. A61B 6/03

(52) U.S. Cl. 600/431; 378/4

(57) ABSTRACT

A surgical positioning device comprises a main part and at least three fiducial markers. Said fiducial markers have a CT number ranging from 1,000 to 3,000 and may produce dense, bright, and artifact-free spots on a CT scanning image.

### BACKGROUND OF THE INVENTION

1. Field of the Invention

The present invention relates to a surgical positioning device used in image guided surgery. More particularly, the invention relates to a surgical positioning device comprising Computed Tomography (CT) fiducial markers with desirable accuracy and no severe artifact.

2. Description of the Related Art

Image guided surgery is a process by which a surgeon performs an operation under the guidance of a three dimensional volumetric image representing the anatomy of a patient. Compared with the conventional un-guided surgery, the image guided surgery is more reliable in that it provides the benefits of minimal invasiveness and high accuracy.

Among the various image modalities, CT is one of the most popular modalities in image guided surgery. Generally, the first step of CT-guided surgery is the collection of CT scan data, which is done by passing a patient through a CT machine pre-operatively; after that, the data is used intro-operatively to provide a surgeon valuable guidance when he/she is to place a surgical device inside the patient's anatomy, which allows he/she seeing or navigating through the patient's anatomy in real time before and during the surgery.

In the CT-guided surgery, the key procedure that makes the image guidance possible is the registration step, which represents the association of the CT scan data taken pre-operatively and that of the patient lying on the operation table during the surgery. Usually, the registration is achieved by attaching at least three fiducial markers to a patient's anatomy during CT scanning. Since the markers may be clearly shown on the CT image in the form of bright spots, they can be used to define a reference coordinate system in the image space. In the meantime, during the surgery, a robot system, a localization device, or an optical navigating system may be used to measure the location of the fiducial markers, which can be used to define another reference coordinate system in the physical space. Therefore, a transformation matrix can be calculated to bring the image space and physical space together by mathematically matching the two defined coordinate systems.

Since the accuracy of the positions of the fiducial markers account for a great weight in the quality of the CT-guided surgery, selecting a fiducial marker capable of demonstrating a desirable property in the image space and the physical space is important. Conventionally, steel balls are commonly used in scanning due to their availability; however, the inherent “metal artifact” inevitably interferes the clarity of the image and deteriorates the quality thereof. In some situations, the artifact is so severe that it not only causes marker image identification problems but also obstructs the subsequent diagnosis.

Accordingly, many efforts have been made to solve the artifact problem. U.S. Pat. No. 5,636,255 describes a method using a reduced-sized metal ball to alleviate the artifact effect. U.S. Pat. No. 6,333,971 B2 provides a method which uses an aqueous solution of metal powder to reduce the percentage of the metal material in the marker constitution. Moreover, U.S. Pat. No. 5,415,546 provides a radiopaque composition made by combining a radiopaque material and a binder. However, several unsolved problems keep these inventions from being fully accepted. In the first invention, the artifact remains because of the existence of the metal object in CT scanning. As to the latter two, problems resulted from unequal mixture will always have to be overcome before they can be successfully reduced to practice. Besides, all the three inventions, which largely depend on an empirical approach to find suitable material, fail to provide a systematic way to identify the eligible material.

Therefore, there is a need for a solid material for a CT fiducial marker which may produce a clear CT image under a normal clinical scanning condition. The requirement of the high quality image of the marker includes the artifact-free image and high brightness, which may facilitate the effective marker identification so as to achieve the high accurate registration of CT-guided surgery.

### SUMMARY OF THE INVENTION

An objective of the present invention is to provide a surgical positioning device used in image guided surgery. By using the surgical positioning device of the invention, users may produce satisfactory CT images on which fiducial markers are bright enough but produce no artifact. Instead of utilizing the conventional try-and-error approach, the present invention proposes a rational and systematic way to calibrate the quality of an object under CT analysis.

The surgical positioning device of the present invention comprises a main part and at least three fiducial markers with a CT number ranging substantially from 1,000 to 3,000, wherein the CT number may be derived from the following equations:

$$CT = 1 , 000 \frac{μ - {μ}_{water}}{{μ}_{water}} ;$$

$$μ = ( μ \text{/} ρ ) ρ ; and$$

$$μ \text{/} ρ = {\sum }_{i} {{w}_{i} ( μ \text{/} ρ )}_{i} , where$$

CT represents the CT number;
μ represents the linear attenuation coefficient;
μ_{water} represents the linear attenuation coefficient of water;
(μ/ρ) represents the mass attenuation coefficient;
ρ represents the density;
wᵢ represents the fraction of weight of the i^{th} atomic constituent; and
(μ/ρ)ᵢ is the mass attenuation coefficient of the i^{th} atomic constituent.

Based on the formulation provided above, potential fiducial markers may be identified with ease. For example, aluminum oxide (Al₂O₃), whose estimated CT number is 2,719, demonstrates great suitability as it produces almost no “metal artifact” on CT scan; similarly, silicon nitride (Si₃N₄), which has an estimated CT number of 2,861, shows a desirable result on CT scan as well. The relatively low error percentages (both less than 1%) between the estimated CT numbers and the experimental ones of the aluminum oxide and silicon nitride validate the soundness of the present invention, making it a reliable solution for the fabrication of surgical positioning devices.

The preferable CT number of the fiducial markers of the invention may range substantially from 1,000 to 3,000. More preferably, it falls within the range of 1,500 to 2,500, and, most preferably, 1,750 to 2,250. The surgical positioning device of the present invention may be applied to various fields of image guided surgery, such as dental surgery, orthopedic surgery, and internal surgery. Likewise, the structure of the main part of the surgical positioning device may also be fabricated in a variety of ways; for example, it may be but not limited to a casting of negative impression of the teeth from the cast model of a patient with which an operator may acquire real-time images through a registration procedure that matches the physical space and the image space.

Other objects, advantages, and novel features of the invention will become more apparent from the following detailed description when taken in conjunction with the accompanying drawings.

### BRIEF DESCRIPTION OF THE DRAWINGS

FIG. 1 is a flowchart of the formulation of the present invention.

FIGS. 2 to 6 show the CT images of a hog with the presence of silicon nitride, aluminum oxide, glass, zirconium oxide, and steel, respectively.

FIG. 7 is an enlarged view of FIG. 2 showing a grid of gray square elements.

FIG. 8 is an illustrative diagram of the surgical positioning device of the present invention.

FIG. 9 is a schematic diagram for the surgical positioning device of the present invention under use.

### DETAILED DESCRIPTION OF THE PREFERRED EMBODIMENT

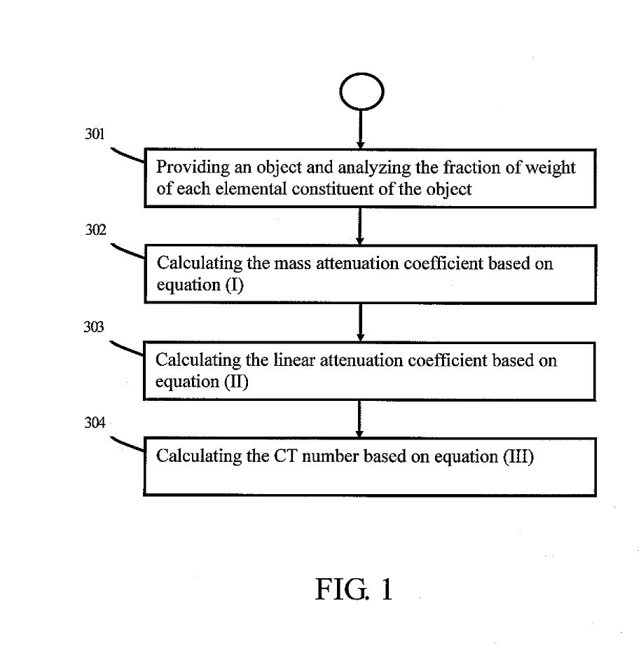

Please refer to FIG. 1 for a flowchart of the formulation of the present invention.
Step 301: Providing an object and analyzing the fraction of weight of each elemental constituent of the object.

First of all, an object of interest is provided with the fraction of weight of each ingredient calculated. Since the analysis of chemical components is a well-established art, further elaboration is omitted hereby.
Step 302: Calculating the mass attenuation coefficient based on equation (I) as follows:

$$μ \text{/} ρ = {\sum }_{i} {{w}_{i} ( μ \text{/} ρ )}_{i} , \qquad equation ( I )$$

wherein wᵢ and (μ/ρ)ᵢ stand for the fraction of weight and the mass attenuation coefficient of the i^{th} atomic constituent respectively.
Step 303: Calculating the linear attenuation coefficient based on equation (II) as follows:

μ=(μ/ρ)ρ equation (II),

wherein μ is the linear attenuation coefficient; ρ is the density of the object.
Step 304: Calculating the CT number based on the equation (III) as follows:

$$CT = 1 , 000 \frac{μ - {μ}_{water}}{{μ}_{water}} , \qquad equation ( III )$$

wherein μ^{water} is the linear attenuation coefficient of water.

Because (μ/ρ) is documented in U.S. National Institute of Standard and Technology (NIST) for elemental material with atomic number from Z=1 to Z=92, the CT number of the object may be obtained by the equations aforementioned. For the material with high mass attenuation coefficient and high density, regardless of elemental material or compounds, it leads to high linear attenuation coefficient according to equation (II) and subsequently renders image pixel of a large CT number. Take steel material for example (μ=0.3717 (cm²/g), p=7.87 (g/cm³)). At the normal clinical radiation energy setting 120 keV, the CT number is estimated as 16,146 from equations (II) and (III), which well exceeds the CT normalization range of −1000 to 3000 that can be handled by the computer. This over-ranging situation leads to incomplete attenuation profiles that may confuse the CT reconstruction algorithm, causing a severe streaking artifact known as “metal artifact”.

The streaking artifact caused by the presence of a metal object in the CT scan field is in a star-burst shape, emanating from the center of the metal object. The range of artifact area depends on the attenuation property and the size of the metal object. Basically, the higher the atomic number of the metal is, the severer the artifact may be produced. By the same token, the area of the artifact may increase when a large-sized metal is used. Therefore, when a metal fiducial marker is used in CT guided surgery, the artifact not only disturbs the clinical diagnosis of surrounding tissues but also presents great difficulties in the identification of the geometrical shape of the marker itself; thus, the use of the highly attenuating material as a CT fiducial marker should be avoided.

On the other hand, the use of low attenuating material as the CT fiducial maker may reduce or even eliminate the artifact; but it may not be radio-opaque enough to make its CT image distinguishable from the soft tissue. In such cases, the purpose of the CT fiducial marker cannot be obtained. In the clinical CT diagnosis, the bone tissue has the largest CT number; therefore, the optimal material for the maker should have a comparable X-ray attenuation property to that of the bone tissue. Such bone-like materials as the CT fiducial marker can provide artifact-free image while the image is bright enough to make the marker identification process efficient.

In one embodiment of the present invention, some physical properties of several materials, together with a table showing their estimated CT numbers and experimental CT numbers, are provided. In addition, the CT image of each material is shown so that the corresponding artifact effect may be observed.

In another embodiment of the present invention, a surgical positioning device comprising a main part and three fiducial markers which have a CT number between 1,000 to 3,000 is disclosed.

Table 1 lists the X-ray attenuation property and the atomic mass of some fundamental elements constituting selected materials for the fiducial marker. It should be noted that the mass attenuation coefficient is related to the radiation energy; and 120 keV is chosen because it is one of the most commonly used radiation energy settings for CT clinical diagnoses.

TABLE 1
X-ray Mass Attenuation Coefficient and Atomic Mass of Some
Fundamental Elements

| | | | Mass |
|---|---|---|---|
| | | | Attenuation |
| | | Atomic | Coefficient |
| Element | Symbol | Mass | (μ/ρ), cm²/g |
| Hydrogen | H | 1.008 | 0.283 |
| Nitrogen | N | 14.008 | 0.146 |
| Oxygen | O | 15.999 | 0.148 |
| Aluminum | Al | 26.982 | 0.157 |
| Silicon | Si | 28.085 | 0.168 |
| Potassium | K | 39.099 | 0.204 |
| Calcium | Ca | 40.078 | 0.232 |
| Iron | Fe | 55.847 | 0.302 |
| Yttrium | Y | 88.905 | 0.687 |
| Zirconium | Zr | 91.224 | 0.731 |

Table 2 lists the CT numbers, both estimated and experiments ones, of some materials along with the reference material water.

TABLE 2
CT Numbers of Selected Element Material and Compounds

| | | Mass | | | |
|---|---|---|---|---|---|
| | | Attenuation | | | Experi- |
| | | Coefficient, | | Estimated | mental |
| | | cm² /g | Density, | CT | CT |
| Material | Constitution | $( {\sum }_{i} {{w}_{i} ( μ / ρ )}_{i} )$ | g/cm³ | number | number |
| Water | H₂O (100%) | 0.163 | 1.00 | 0 | — |
| Silicon | Si₃N₄ (92%) | 0.190 | 3.30 | 2,861 | 2.882 |
| nitride | Y₂O₃ (8%) | | | | |
| Alumi- | Al₂O₃ (99.9%) | 0.153 | 3.96 | 2,719 | 2,716 |
| num | | | | | |
| oxide | | | | | |
| Glass | SiO₂ (70%) | 0.170 | 2.60 | 1,723 | 1,893 |
| | CaO (15%) | | | | |
| | K₂O (15%) | | | | |
| Zirco- | ZrO₂ (97%) | 0.580 | 3.96 | 19,501 | severe |
| nium | unknown (3%) | | | | artifact |
| oxide | | | | | |
| Steel | Fe (100%) | 0.302 | 7.87 | 13,602 | severe |
| | | | | | artifact |

To verify the effectiveness of the formulation on the CT number estimation, experimental CT scanning on the selected materials were carried out using a clinical CT machine (GE, LightSpeed VCT model). By comparing the estimated and experimental CT numbers as listed in Table 2, it can be concluded that the formulation of the CT number estimation for compounds as devised in this invention is effective. The formulation successfully estimates the CT number with accuracy higher than 90% for the moderate attenuating materials, and more remarkably, with accuracy higher than 99% for silicon nitride and aluminum oxide, two materials possessing desirable properties. It should be noted that even though zirconium oxide (ZrO₂) contains 3% unknown material, its influence on the CT number can be ignored due to the main contribution of the large CT number resulting from 97% of ZrO₂.

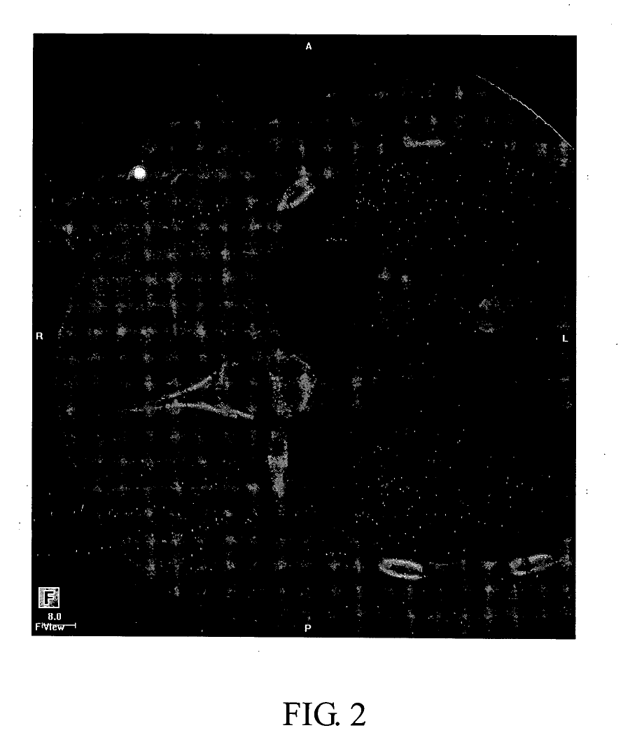

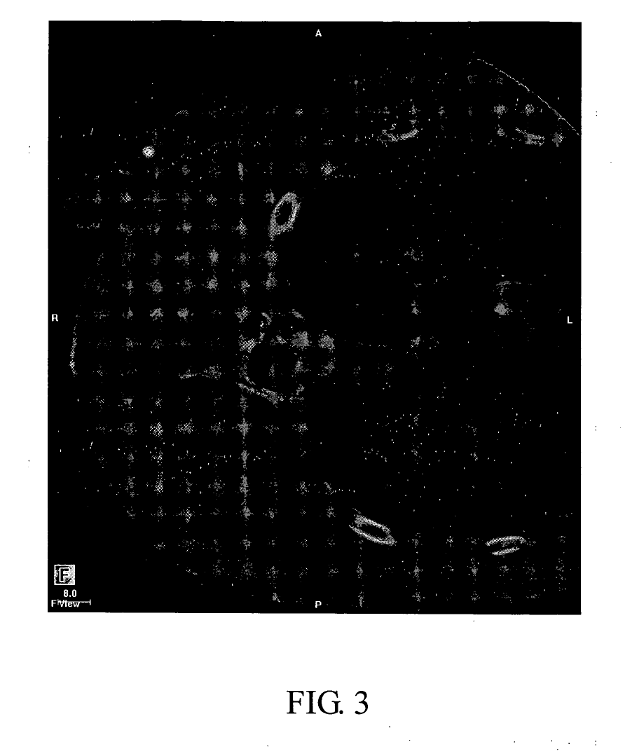

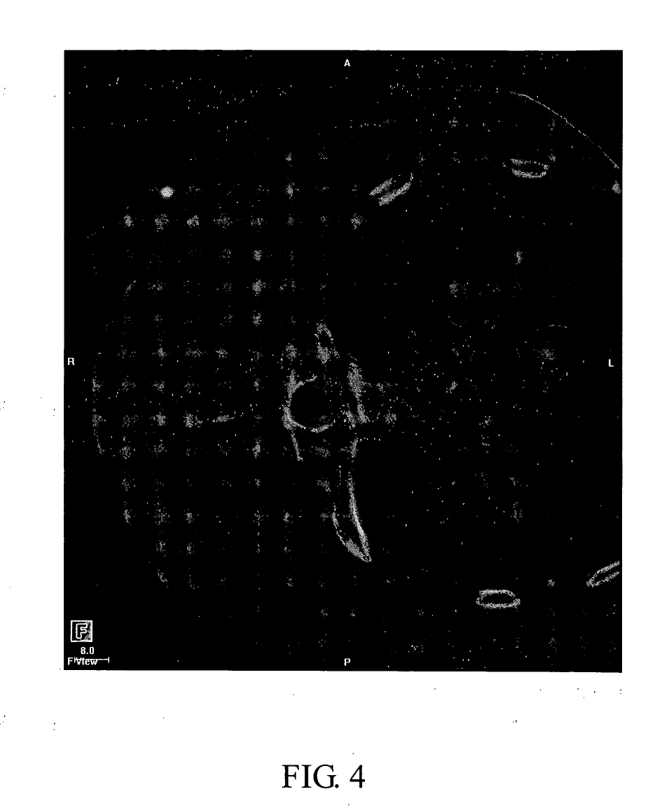

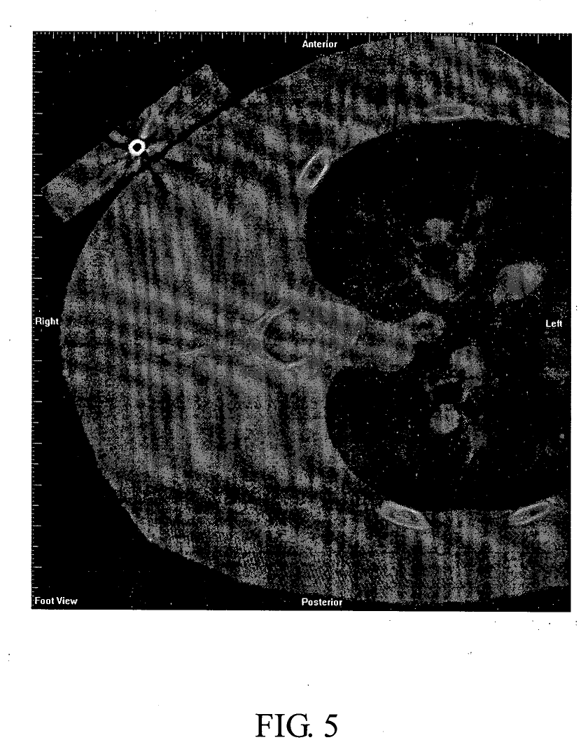

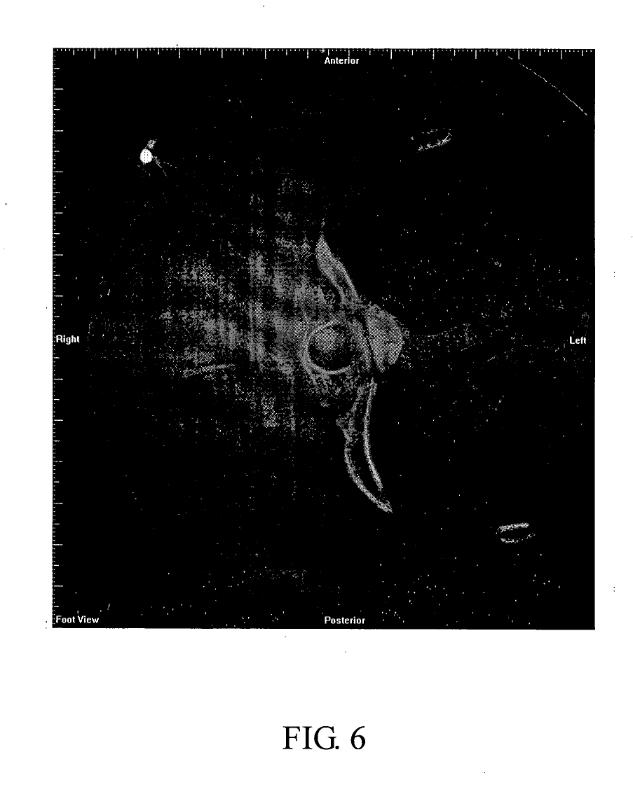

Refer now to FIGS. 2 to 6 for the CT images of a hog in the presence of various 3 mm balls with the material of silicon nitride, aluminum oxide, glass, zirconium oxide, and steel, respectively. It can be clearly shown that, in line with what the estimated CT numbers foreshadow, the first three materials exhibit bright, dense, and artifact-free spots on the images, whereas zirconium oxide and steel present serious interferences. Therefore, it is fair to say that the first three materials—silicon nitride, aluminum oxide, and glass—may serve as good fiducial markers for CT guided surgery.

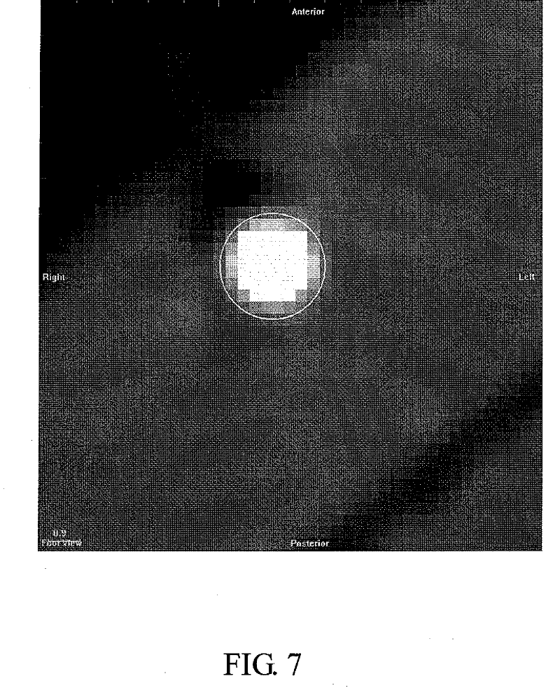

FIG. 7 is an enlarged picture of FIG. 2. In FIG. 7, the portion around the fiducial marker is magnified to such an extent that, due to the finite resolution of the CT scanner, the image of the fiducial marker is represented by a grid of gray square elements know as “pixels,” whose grayscales are termed as the CT number. Although the fiducial marker is uniform, the CT number gradually decreases from the center to the boundary because of the “partial volume effect.” Thus, to find out the CT number of the marker from the blurring image, a circle with the physical diameter of the ball, centering at the center of the marker, is drawn. Then the CT number of each corresponding pixel inside the circle is summed into an averaged CT number.

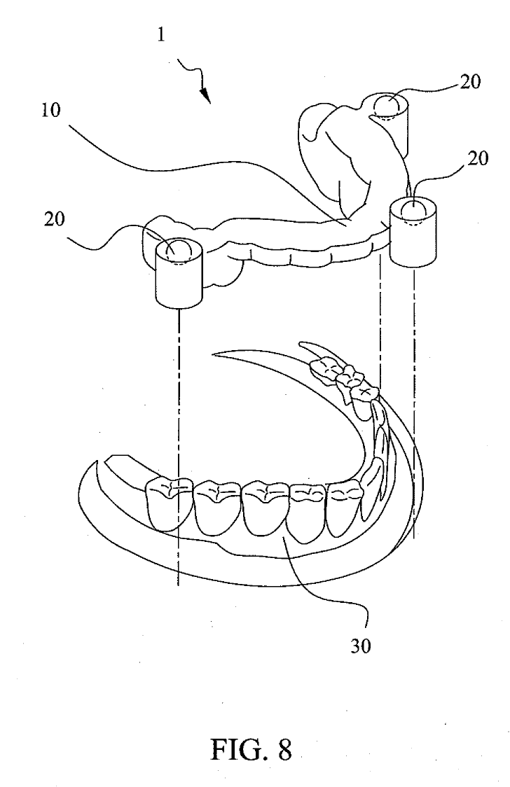

Refer now to FIG. 8 for the second embodiment of the invention. As shown, the present invention provides a surgical positioning device 1, comprising a main part 10 and at least three fiducial markers 20, wherein said main part 10 being a casting of negative impression of the teeth 30 of a patient, and the fiducial markers 20 having a CT number substantially of 1,000 to 3,000. It should be noted that the main part 10 may also be but not limited to other types of wearable articles that may be mounted on a patient. The fiducial markers 20 may be any material having a CT number substantially of 1,000 to 3,000, more preferably, 1,500 to 2,500, or most preferably, 1,750 to 2,250. Certainly, as shown in Table 2 and FIGS. 2 to 4, silicon nitride, aluminum oxide, or glass may be suitable material for the fiducial markers 20.

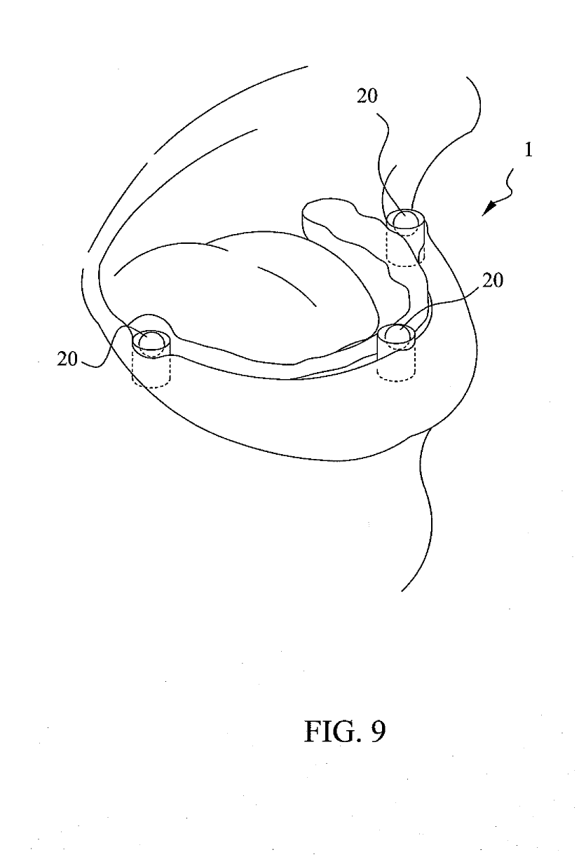

FIG. 9 illustrates the surgical positioning device 1 of the present invention under use. To conduct image guided surgery, for example, a surgeon first mounts the surgical positioning device 1 on a patient's teeth and makes him/her pass through a CT machine so as to collect CT scan data therefrom. Afterward, a registration step is performed by associating the CT scan data with the positions of the three fiducial markers 20. By the application of the fiducial markers 20 with a desirable CT number, the reliability and precision of the surgery may be raised accordingly, directly validating the soundness of this invention.

Although the present invention has been explained in relation to its preferred embodiments, it is to be understood that many other possible modifications and variations can be made without departing from the spirit and scope of the invention as hereinafter claimed.

1. A surgical positioning device, comprising:
a main part; and
at least three fiducial markers with a CT number substantially of 1,000 to 3,000 mounted on the main part, wherein the CT number is derived from the following equations:
$$CT [ [ • ] ] \underline{=} 1 , 000 \frac{μ - {μ}_{water}}{{μ}_{water}} ;$$
$$μ [ [ • ] ] \underline{=} ( μ \text{/} ρ ) ρ ; and$$
$$μ \text{/} ρ [ [ • ] ] \underline{=} {\sum }_{i} {{w}_{i} ( μ \text{/} ρ )}_{i} , where$$
CT represents the CT number;
μ represents the linear attenuation coefficient;
μ_{water} represents the linear attenuation coefficient of water;
(μ/ρ) represents the mass attenuation coefficient;
ρ represents the density;
wᵢ represents the fraction of weight of the i^{th} atomic constituent; and
(μ/ρ)ᵢ is the mass attenuation coefficient of the i^{th} atomic constituent.

2. The surgical positioning device as claimed in claim 1, wherein the CT number ranges substantially from 1,500 to 2,500.

3. The surgical positioning device as claimed in claim 1, wherein the CT number ranges substantially from 1,750 to 2,250.

4. The surgical positioning device as claimed in claim 1, wherein the fiducial markers essentially consist of silicon nitride (Si3N4).

5. The surgical positioning device as claimed in claim 1, wherein the fiducial markers essentially consist of aluminum oxide (Al2O3).

6. The surgical positioning device as claimed in claim 1, wherein the fiducial markers essentially consist of glass (SiO2).

7. The surgical positioning device as claimed in claim 1, wherein the fiducial markers are beads.

8. A fiducial marker having a CT number substantially of 1,000 to 3,000, wherein the CT number is derived from the following equations:
$$CT [ [ • ] ] \underline{=} 1 , 000 \frac{μ - {μ}_{water}}{{μ}_{water}} ;$$
$$μ [ [ • ] ] \underline{=} ( μ \text{/} ρ ) ρ ; and$$
$$μ \text{/} ρ [ [ • ] ] \underline{=} {\sum }_{i} {{w}_{i} ( μ \text{/} ρ )}_{i} , where$$
CT represents the CT number;
μ represents the linear attenuation coefficient;
μ_{water} represents the linear attenuation coefficient of water,
(μ/ρ) represents the mass attenuation coefficient;
ρ represents the density;
wᵢ represents the fraction of weight of the i^{th} atomic constituent; and
(μ/ρ)ᵢ is the mass attenuation coefficient of the i^{th} atomic constituent.

9. The fiducial marker as claimed in claim 8, wherein the CT number ranges substantially from 1,500 to 2,500.

10. The fiducial marker as claimed in claim 8, wherein the CT number ranges substantially from 1,750 to 2,250.

11. The fiducial marker as claimed in claim 8, wherein the fiducial marker essentially consists of silicon nitride (Si3N4).

12. The fiducial marker as claimed in claim 8, wherein the fiducial marker essentially consists of aluminum oxide (Al2O3).

13. The fiducial marker as claimed in claim 8, wherein the fiducial marker essentially consists of glass (SiO2).

14. The fiducial marker as claimed in claim 8, wherein the fiducial marker is a bead.
